(19) United States
(12) Patent Application Publication
(10) Pub. No.: US 2015/0104503 A1
(43) Pub. Date: Apr. 16, 2015

(54) COMPLEXES AND COMPOSITIONS CONTAINING CURCUMIN

(71) Applicant: Harold Gordon CAVE, Hamilton (NZ)

(72) Inventor: Harold Gordon Cave, Hamilton (NZ)

(21) Appl. No.: 14/402,071

(22) PCT Filed: May 22, 2013
(86) PCT No.: PCT/NZ2013/000086

§ 371 Date: Nov. 18, 2014

(30) Foreign Application Priority Data
May 22, 2012 (NZ) 600146

(51) Int. Cl. A61K 47/48; A61K 35/56; A61K 31/353; A61K 31/202; A61K 31/12; A61K 31/4525

(52) U.S. Cl. CPC A61K 47/48053; A61K 31/12; A61K 31/4525; A61K 31/353; A61K 31/202; A61K 35/618
USPC 424/451; 514/78; 554/80; 424/522

(57) ABSTRACT

A complex is provided, including a phospholipid and curcumin, wherein the phospholipid is sourced from a marine oil.

### TECHNICAL FIELD

The present invention relates to improvements in and relating to the bioavailability of curcumin, and methods of producing complexes and compositions providing improved bioavailability of curcumin.

### BACKGROUND ART

Curcumin is a compound present in the spice turmeric. Curcumin has been shown in many studies to have pharmacologic effects such as antioxidant, anti-inflammatory, antiproliferative and antiangiogenic activities. As such, curcumin represents a target to fight diseases such as cancer, heart disease diabetes, Crohn's disease and various neurological diseases. For this reason, there has been significant research on curcumin over the past 20-30 years.

A significant advantage of curcumin is its wide acceptance due to it being a natural compound used for centuries as a spice in food such as curries. A further advantage is that, even at high doses, there are little to no side effects. It is also relatively cheap to source, and stores well at room temperature.

Despite these advantages, an overriding issue which has yet to be addressed is curcumin's well known problem of low bioavailability in animals. This is thought to be due to a combination of factors including poor solubility and hence poor absorption, elimination from the system and/or quick metabolism.

In the past, this poor solubility has been overcome, at least in in vitro studies, is by adding carriers such as DMSO or Tween 80 which help to increase solubility of the curcumin. However, addition of these carriers in a therapeutic medicament would not be suitable reasons, primarily because carriers such as DMSO lead to a foul taste, it adds to the manufacturing cost and process, and detracts from the advantage that curcumin is a natural product (which consumers like).

Combining curcumin with an oil can improve the uptake of curcumin into the systemic system. However, because the curcumin does not bind with the oil it drops out of suspension after mixing. Agitating the curcumin and oil mixture vigorously can provide a slightly improved product due to a small percentage being solubilised. However, the shelf life is limited as the curcumin will experience sedimentation over time. Regardless of how vigorously the curcumin and oil mixture is mixed, centrifuging the product will effectively separate most of the curcumin and oil.

In order to try to overcome the poor absorption/stability issue and to maximise from curcumin's beneficial effects, numerous approaches have been investigated over the past few decades. These include preparation of liposomal or phospholipid structures, nanoparticles, and structural analogues. Anand et al., Mol. Pharmaceutics, 2007, 4 (6), 807-818 provides a good review of these different approaches.

For example, WO 2007/101551 describes a phospholipid complex with curcumin using lipids from vegetable or synthetic origin. A high molar ratio of curcumin to lipid was provided having about 16.9% curcumin in the resulting complex. However, the resulting product was a viscous wax. This would make it substantially impossible to encapsulate, and hence the product would almost certainly be provided in a tablet form. Although tablets are a suitable form for delivery of the complex, from the manufacturing perspective, encapsulation can be a more attractive option particularly for oil based formulations. Encapsulation is often only readily achievable if the resulting complex solution is not too viscous.

A different avenue many research groups are exploring is combining curcumin with adjuvants. Compounds like piperine, quercetin or Omega-3 polyunsaturated fatty acids, such as docosahexaenoic acid (DHA) and/or eiscosapentaenoic acid (EPA) have recently been shown to produce a synergistic therapeutic effect when used in combination with curcumin, although the exact modes of action are still uncertain. These approaches have also been outlined in Anand et a/, 2007.

It is also thought that the synergy of curcumin is limited to a relatively small subset (about 8) of polyunsaturated fatty acids including DHA and EPA because these fatty acids have carbon chain length between 20 or above. This allows the body to readily absorb them. This is comparable to other fatty acids such as linoleic acid which has a carbon chain length of 18.

For example, Altenburg et al., BMC Cancer 2011, 11; 149 describes the synergy of DHA and curcumin in inhibiting numerous breast cancer cell lines. It was herein reported that the optimum ratio of DHA to curcumin was approximately 2:1 to 1:1, depending on the type of breast cancer cell line being inhibited.

In a further study, Swamy et al., Nutrition and Cancer 2008, 60: S1, 81-89 reported that a ratio of about 2.5:1 (DHA to curcumin) showed the greatest effect on apoptosis in BxPC-3 cells, a form of pancreatic cancer cells.

Therefore, although there is reasonable information guiding the formulator to optimal ratios of curcumin to adjuvants such as DHA, it can often be difficult to formulate a composition retaining the desired ratios (which may differ depending on the exact therapeutic effect desired), whilst also trying to accommodate for the issues around instability, insolubility and poor absorption of curcumin, as well as other factors of the composition such as preferred viscosity requirements.

For example, in attempting to achieve a desired molar ratio (and concentrations) between DHA and curcumin, it can lead to a higher viscosity which can similarly make encapsulation a difficult option for manufacturing.

Therefore, despite certain advances, there is still significant need to improve on the bioavailability and therapeutic effect of curcumin, the ability to increase loading of curcumin, the stability of resulting compositions, as well as the ease/cost of manufacturing the medicament in a convenient dosage form.

It is an object of the present invention to address one or more of the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### DISCLOSURE OF THE INVENTION

According to a first aspect of the present invention there is provided a complex including a phospholipid and curcumin,

characterised in that the phospholipid is sourced from a marine oil.

According to a further aspect of the present invention there is provided a complex including a phospholipid and curcumin,

characterised in that the phospholipids are sourced from a marine oil and a lecithin.

According to a further aspect of the present invention there is provided a composition including a complex with a phospholipid and curcumin,

characterised in that the phospholipid in the complex is sourced from a marine oil.

According to a further aspect of the present invention there is provided a method of preparing a complex or composition substantially as herein described above

the method including the steps of:
a) forming a first solution by dissolving a quantity of curcumin in a solvent;
b) forming a further solution by mixing the first solution with a quantity of the phospholipid sourced from a marine oil
c) processing the further solution to form the complex
d) separating the complex from the solvent.

According to a further aspect of the present invention there is provided a method of treatment using the composition substantially as herein described above, wherein the composition is used to treat or prevent, or at least provide complementary treatment or prevention, to one of the following conditions:
cancer,
heart disease
diabetes,
Crohn's disease and
various neurological diseases.

The present invention surprisingly and advantageously benefits from the clever use of phospholipids from marine oils that have a high content of phospholipids and are naturally enriched with polyunsaturated fatty acids such as DHA and EPA. In brief, the present invention:
allows formation of a stable complex using phospholipids from the marine oil, and by forming a stable, complex, prevents separation of the components (therefore the present invention ensures effective absorption of the curcumin)
provides a high level of phospholipids, and in turn a high level of polyunsaturated fatty acids such as DHA and EPA (inherently present as in the tails of the marine oil phospholipids). This increased level of fatty acids therefore helps to achieve a desired molar ratio of curcumin to fatty acids, which in turn is beneficial to provide the synergistic effect seen between these fatty acids with curcumin.

Throughout this specification the term marine oil should be taken as meaning any oil which is sourced from sea-based organisms such as fish and shellfish and wherein the oil includes a phospholipid or phospholipids containing at least one type of polyunsaturated fatty acid.

Some specific examples of such marine oils include mussel oil, krill oil, salmon oil, squid oil and so forth. Another example of marine oils may be the oil from roe, again perhaps from fish or shellfish.

Marine oils such as these exemplified above beneficially have relatively high phospholipid content. Therefore these may be beneficially used for the present invention to help achieve a preferred molar ratio of curcumin to phospholipids and fatty acids, as discussed further below. By using marine oils such as those exemplified above, the complex also beneficially provides the synergistic therapeutic effect offered by the Omega-3 fatty acids such as DHA or EPA with the curcumin present in the marine oil sourced lipids.

It should be appreciated that it is possible that the phospholipids are first extracted from the marine oil (for instance through acetone precipitation) and then subsequently added to the curcumin (minus the remaining components of the marine oil) to form the complex. In such a case, a waxy substance may result after precipitation of the phospholipids, which may then be thinned down with a diluent before being complexed with the curcumin.

Preferably, the composition includes marine oil. This is a preferred option because it is possible many of the components of the marine oil may be improving the therapeutic effectiveness or stability of curcumin. For example, mussel oil has about 91 different types of fatty acids.

Despite this synergy between the fatty acids DHA and/or EPA with curcumin being known for considerable time, no one in the industry has yet arrived at the present invention of actually complexing the curcumin with a marine oil sourced phospholipid. Up until now, researchers have only been demonstrating a combination of DHA/Omega 3 with curcumin, but have never thought to actually complex the curcumin with the phospholipid which are rich in such components, thereby achieving two beneficial effects with a single component (namely benefiting from the synergy between the fatty acids, and also forming the complex with the phospholipids).

On the other hand, researchers have primarily turned to using vegetable oils such as soyabean lecithin oil to complex curcumin. It is thought that soy phospholipids have been the mainstay for this curcumin complexing because they are known to be highly absorbed in humans, and do not show any chronic effects on animals in vivo, even at high dosages. Also the high amounts of polyunsaturated fatty acids like linoleic acid present in soyabean oil has made it an attractive option for reducing the risk of diseases like heart disease. Yet, a major disadvantage is that there is no reported synergy with shorter chain fatty acids in vegetable oils (like linoleic acid) and curcumin.

Throughout this specification the term phospholipid should be taken as meaning any type of lipid that includes a hydrophobic tail and hydrophilic head. Phospholipids, in context of this invention, are used to form micellar complexes to protect the curcumin.

Preferably, the marine oil contains greater than 20% w/w phospholipid. Marine oils such as salmon oil, mussel oil, krill oil and squid oil are all known to have phospholipid contents above 20%. One particularly preferred marine oil with high levels of phospholipid is mussel oil, with levels of about 65% w/w phospholipid.

Most preferably, the marine oil contains approximately 40% w/w phospholipid.

More preferably, the phospholipid is selected from the group consisting of phosphatidylcholine (PC), phosphatidic acid (PA), phosphatidylethanolamine (PE), phosphatidylserine (PS), phosphatidylinositol (PI), phosphatidylinositiol phosphate (PIP), phosphatidylinositiol biphosphate (PIP2), and phosphatidylinositiol triphosphate (PIP3).

Most preferably, at least a portion of the phospholipid in the complex is phosphatidylcholine (PC). This is because PC is a commonly used phospholipid and is well understood in the industry. However, potentially any one or combination of phospholipids could be used with the present invention.

Again, good examples of marine oils that have both PC and PI include mussel oil, krill oil and salmon oil.

Alternatively, the phospholipid is selected from the broad class of phosphosphingolipids.

In this alternative embodiment, the phospholipid is selected from the group consisting of ceramide phosphorylcholine or ceramide phosphorylethanolamine (Sphingomyelin, SPH or Cer-PE respectively) and ceramide phosphorylipid.

Throughout this specification the term curcumin should be taken as meaning any curcuminoid. A curcuminoid may simply be curcumin as shown in the structure below, or may be a derivative of curcumin with varied chemical groups which provide improved stability or other pharmacokinetic properties of the compound.

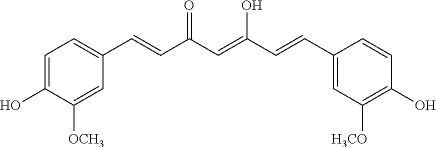 [SMILES: COc1cc(/C=C/C(=O)/C=C(O)/C=C/c2ccc(O)c(OC)c2)ccc1O]

The curcumin may be isolated from a natural source such as turmeric, or it may be synthetically prepared through a range of techniques.

Most preferably, demethoxycurcumin is used. In Cuomo et al, J. Nat. Prod. 2011, 74 664-669, it was shown that phospholipid formulation increased the absorption of demethoxylated curcuminoids much more than that of curcumin, therefore making it particularly applicable for use in the current invention. A further commonly used alternative curcumin is bisdemethoxycurcumin.

However, any other form of curcumin, either known currently or developed in future, may be used as part of the present invention without departing from the scope thereof.

Preferably, the complex includes above 1% w/w curcumin.

Preferably, the complex includes between 1 to 15% w/w curcumin.

As discussed previously, a higher concentration and loading of curcumin is one strategy to improve the ultimate aim of greater absorption of curcumin into the body.

More preferably, the complex includes approximately between 2-8% w/w curcumin. It would certainly be possible to increase the level of curcumin beyond 8% within the complex of the present invention. This is discussed further in the specification.

### Preferred Example of Molar Ratios in the Complex

Most preferably, the molar ratio of the curcumin to polyunsaturated fatty acids in the complex is in the range of about 1:2 to 20:1.

Most preferably, the molar ratio of the curcumin to polyunsaturated fatty acids in the complex is in the range of about 1:2 to 5:1.

As discussed previously with reference to Altenburg and Swamy, there are reports to show that the most preferred molar ratio which provides the most effect synergy for the cancer cell lines studied was about 2.5 to 1 (DHA to curcumin). However it is clear a higher ratio upwards of 20:1 (DHA and/or other fatty acids such as EPA to curcumin) also show synergy, but to a lesser degree. It quite possible that depending on the particular therapeutic use (for example, the type of cancer cells to be targetted), the molar ratio that provides a heightened synergy could vary.

Most preferably, the ratio of curcumin to phospholipid sourced from the marine oil is approximately between 1:5 and 1:20.

Such preferred ratios are beneficial as they are seen to effectively work with the trial examples performed by the inventor and are well documented to provide suitable complexing of curcumin with the phospholipid. For example, WO 2007/101551 provides shows in Example 4 that up to 16% w/w curcumin could be achieved in a stable complex when the ratio of curcumin to phospholipid is as high as 1:4. Obviously, one could achieve a stable complex if the amount of phospholipid is increased in this ratio.

An example of how a particularly preferred 1:10 ratio of curcumin to phospholipids is provided below.

Mussel oil has approximately 65% w/w phospholipids, and also contains about 24% w/w EPA and 13% DHA, totalling about 37-38% polyunsaturated fatty acids which is predominantly bound to the phospholipids. Therefore, if one were to add 4 kg of curcumin to 100 kg (approximately 100 L) of mussel oil, a ratio of approximately 1:16 (curcumin to phospholipid) may be achieved in the resulting complex, and provide about a 40 g/L (or 4% w/w) curcumin in the complex.

Research shows that the therapeutic effect of curcumin in the body (suggested by in vitro trials) is only about 10 uM (3 mg/L curcumin). Therefore, this composition may provide a significant excess of curcumin, yet much of this may not be absorbed in vivo as previously discussed. This assumes that, based on studies performed by the inventor that close to 100% of the curcumin added is able to complex with the phospholipids.

Similarly, this example would provide a molar ratio of about 11:1 fatty acids (namely DHA and EPA combined) to curcumin. The specific molar ratio of DHA to curcumin would be about 4:1, and 8:1 for EPA to curcumin. As noted above, this is within the exemplified preferred molar ratio of DHA to curcumin shown to provide a synergistic effect. However, it would still be preferable to increase these molar ratios, for example to about 2.5:1 DHA to curcumin which is shown to have the most effective synergy.

For example, this most preferred molar ratio may be achieved simply by increasing the amount of curcumin in the complex to about 12% w/w (ie bringing the ratio to DHA close to 2.5:1). This is achievable as there is “room to move” within the preferred ratio of curcumin to phospholipids (provided by the mussel oil) required to achieve a stable complex. However, in doing so, the composition may become quite viscous or waxy due to high amount of fatty acids in the mussel oil sourced phospholipids. Although this may be difficult to provide in capsule form with an oil based liquid, one could still provide this in tablet form due to the higher viscosity.

### Preferred Embodiments of a Complex Including Lecithin

To help achieve/and or maintain the desired molar ratio of curcumin to fatty acids, and also help avoid the problem with an increased viscosity which may happen as a result of the fatty acids, the inventor has arrived at a particularly preferred and inventive concept. This is a particularly preferred concept for the encapsulation of a lower viscosity oil based liquid containing the complex and/or complex of the present invention.

Preferably, the complex includes an amount of lecithin.

Throughout this specification the term lecithin should be taken as meaning any mixture of substances from animal or plant tissue which includes phospholipids together with other components such as phosphoric acid, choline, fatty acids, glycerol, glycolipids, and/or triglyerides. The term lecithin throughout this specification should similarly be understood that it is a different substance to the marine oil of the present invention and one which provides a source of phospholipids but is devoid of Omega-3 fatty acids such as DHA and EPA.

In such embodiments, it should be understood that the overall amount of phospholipids may remain the same (e.g. preferably between 1:100 to 1:5 curcumin to phospholipids), wherein the lecithin is providing some of the phospholipids for this preferred ratio to curcumin.

The inventor found that when incorporating lecithin, the stability of the complex with the phospholipids and the curcumin was not hampered as the molar ratio of curcumin to phospholipid does not overly change.

Similarly, the amount of curcumin may be increased in the complex and composition supported by the phospholipids from the lecithin and from the marine oil phospholipids.

As a result, it allows the molar ratio of the curcumin to fatty acids (from the marine oil phospholipids) to be increased within the complex, because the amount fatty acids is not increased in the complex. Not only does this allow the formulator to increase the molar ratio of curcumin to fatty acids, but it also avoids the deleterious increases in viscosity attributed to the fatty acids.

Therefore, the inclusion of lecithin cleverly still provides the necessary source of phospholipids for the complex's stability and protection of curcumin, but without a disadvantage of adding additional Omega 3 fatty acids (namely increased viscosity). Hence, the result of adding lecithin is that one may advantageously increase the molar ratio of Curcumin to fatty acids to the desired level without overly affecting the viscosity of the complex and/or composition.

Preferably, the ratio of lecithin to marine oil in the composition is between 1:3 to 3:1. Most preferably, the ratio of lecithin to marine oil in the composition is approximately 2:1.

In a preferred embodiment, the lecithin is a vegetable lecithin oil. For example, the vegetable lecithin oil may be soybean lecithin oil or sunflower lecithin oil which are considered by the inventor to be particularly applicable to the present invention. This is because both sunflower lecithin oil and soyabean lecithin oil provide choline as a health supplement for brain function.

However, other sources of lecithin are also within the scope of the present invention.

The inventor foresees that the lecithin is best provided in a liquid form opposed to a powdered form to avoid disadvantageously and unnecessarily increasing the viscosity.

Preferably, the composition includes a diluent.

The inventor foresees that the amount of phospholipid provided by the marine oil and lecithin is sufficient to support a stable complex with a concentration of curcumin up to 5% w/w and potentially higher as documented in WO 2007/101551.

Yet, at these higher concentrations of curcumin above about 5% w/w, even with lecithin added to the composition in place of portion of the marine oil phospholipids, the viscosity of the complex may increase beyond what is practical to encapsulate.

Preferably, the composition includes a diluent. This is a particularly advantageous feature beyond the addition of lecithin because one can more easily increase the concentration of curcumin in the complex to concentrations discussed in WO 2007/101551 whilst keeping the viscosity at a practical level for encapsulation, and whilst also beneficially achieving the desired molar ratio of curcumin to fatty acids. This clever approach relies on the knowledge and preliminary tests performed by the inventor that due to the ability to push the ratio of curcumin to phospholipids upwards of 1:5, the phospholipid amount may be easily provided by either the mussel oil or the lecithin. Therefore, by replacing an amount of the lecithin (for example one part lecithin) with diluent, the overall viscosity of the composition may be lowered, but still keep the complex stable.

For example, an approximate ratio of 1:1:1 (diluent:lecithin:mussel oil) is envisaged by the inventor. However, it should be appreciated that alternative ratios or amounts of diluents to lecithin and mussel oil may be used as long as the formulator keeps the curcumin stable in a complex. This would be straightforward to identify through simple trials and therefore should not be considered beyond the scope of the invention.

Preferably, the diluent is oleic acid. The advantage of a diluent such as oleic acid is it provides the same oil base as the remainder of the composition, but advantageously lacks phospholipids and therefore is of lower viscosity to the other components. Of course, other types of diluents may be possible, but the inventor prefers those which do not include those rich in Omega 6, as these have been linked to heart disease.

Preferably, the viscosity of the composition is below 5000 cP measured at 35° C. on a Spindle 21 at 1.5 rpm.

Preferably, additional components may be incorporated into the composition. These components do not necessarily bind to the complex but may help to improve stability, or for instance, may be added as adjuvants to improve the therapeutic effect of the composition.

Preferably, the composition includes an additional source of Omega 3 fatty acid. For example, DHA may be added separately to the composition. In various scientific studies towards treatment or prevention of cancer, DHA has been shown to synergistically act with curcumin. It is possible that this synergy will transpire to other therapeutic uses of curcumin as well.

Preferably, the composition includes quercetin.

Preferably, the composition includes piperine.

Both quercetin and piperine are known to be adjuvants to curcumin, for example to increase absorption or potency of the curcumin for an improved therapeutic effect.

Preferably, some or all of the adjuvants are simultaneously complexed with the phospholipids. As discussed below, the quercetin and piperine are preferably added together with the curcumin prior to dissolving in the solvent, and prior to the addition of the phospholipids sourced from the marine oil (and preferably the lecithin/diluent in some embodiments). In this way, the quercetin is thought to be able to be complexed with the phospholipids in the same manner as the curcumin.

### Method of Manufacture

According to a further aspect of the present invention there is provided a method of preparing a composition substantially as herein described above

the method including the steps of:
a) dissolving a quantity of curcumin in a solvent to form a first solution;
b) mixing the first solution with a quantity of the phospholipid sourced from a marine oil to form a second solution;
c) processing the second solution to form the complex; and
d) separating the complex from the solvent.

The solvent(s) used in the present invention may vary depending upon the type or amount of curcumin used, and the type or amount of phospholipid and/or other components intended for the composition or complex. Therefore the exact composition of the solvent should not be seen as being limiting.

Preferred embodiments in which the first constituent is a natural plant or animal based extract may utilise one or more solvents from the following list, it should be appreciated however that this list is not exhaustive and therefore should not be seen as being limiting.
Hexane
Benzene
Toluene
Diethyl ether
Chloroform
Acetic acid
Butanol
Isopropanol
Propanol
Ethanol
Methanol
Formic acid
Dimethyl Sulfoxide,
Acetone.

Preferably, the solvent is a Protic solvent. Throughout this specification, the term Protic solvent should be taken as meaning any solvent that has a hydrogen atom bound to an oxygen (i.e. a hydroxyl group) or a nitrogen (i.e. an amine group). From the list above, protic solvents include acetic acid, butanol, isopropanol, ethanol, methanol and formic acid.

Most preferably, the solvent is ethanol.

Preferably step a) includes mixing approximately 40-50 parts volume of solvent to about 1 part curcumin.

This ratio helps to ensure the curcumin is properly dissolved and prevents precipitation during the mixing process. The process may also be aided by performing the dissolving step at warmer temperatures.

Solvents such as ethanol may be advantageous as utilise food grade quality ethanol is commercially available for processing techniques such as this.

The step of processing the second solution to form the complex may be achieved through numerous ways. There are many known techniques to form micellar complexes using phospholipids and a drug or compound, for example as documented in WO 2007/101551. Yet without forming the complex, the curcumin and phospholipid are unstable and will separate quickly. This is not only a problem for shelf-life stability, but it also lowers the bioavailability of the curcumin as noted in the background art. To improve absorption, techniques including forming complexes help to keep the curcumin bound to the phospholipids for improved absorption in the body.

Preferably, step c) includes separation by way of evaporation.

Preferably, step c) includes heating the second solution to raise the temperature of the second solution to greater than the boiling point of the solvent, but less than the remaining components in the second solution.

Preferably, step c) includes heating the second solution in a pressure vessel.

Most preferably, the second solution is heated at below atmospheric pressure. This reduces the boiling point of the solvent and fluid and allowing efficient evaporation at lower temperatures.

In some embodiments the pressure in the pressure vessel may be reduced sufficiently to facilitate evaporation of the solvent at room temperature, thereby eliminating the need for heating.

In some embodiments the pressure in the pressure vessel may be raised to increase the boiling point of the solvent and fluid, thereby requiring a greater level of heating to evaporate the solvent.

It may also be beneficial that steps a) to step c) include a period of refluxing, for example 30 minutes. The refluxing may occur during the process of dissolving the curcumin in solvent, and subsequently after the phospholipids have been added to the solution. During these steps, the ethanol is “boiled away” whilst still contained in the pressure vessel, such that the evaporated ethanol falls back into the solution in a cyclic fashion for this period of time. This refluxing helps in the formation of the complex.

In a particularly preferred embodiment of the method of manufacture, the method includes the steps of:
a) dissolving a quantity of curcumin in ethanol to form a first solution;
b) mixing the first solution with a quantity of a marine oil containing phospholipids to form a second solution, and
c) boiling off the ethanol from the second solution to result in a complex between the phospholipid and curcumin.

In some embodiments the steps a) and b) are conducted simultaneously by combining a curcumin, ethanol and phospholipid sourced from a marine oil together and mixing to form the said second solution.

As a further preferred embodiment, step b) includes adding an amount of lecithin to the first solution. In this embodiment, the most preferred ratio of lethicin to marine oil is approximately 2:1.

As a further preferred embodiment, step b) includes adding an amount of diluent to the first solution. Of course, the addition of lecithin and/or the diluent may be added at a different stage, and is of no significant consequence to the outcome of the invention.

In some preferred embodiments the step of boiling may be performed at atmospheric pressure or above, whereby the further solution is heated in order to exceed the boiling point of the ethanol.

In preferred embodiments the ethanol that is boiled off is collected through an evaporator and recovered.

In some preferred embodiments the step of boiling may be performed by reducing the pressure in a vessel containing the further solution, the reduced pressure lowering the effective boiling point of the ethanol.

Reducing the temperature of evaporation by applying a vacuum is advantageous as it reduces the chance of the oil becoming rancid. Oils are sensitive to heat, light and exposure to oxygen. The use of a vacuum reduces both the contribution of heat and of oxygen to the degeneration of the oil.

Preferably, step c) includes applying a slow vacuum at around 40-50° C. for 1-2 hours and then a full vacuum until all the ethanol is boiled off.

In some embodiments a combination of reduced pressure and heating of the further solution may be employed to evaporate off the ethanol.

Reducing the temperature of evaporation by applying a vacuum is advantageous as it reduces the chance of the oil becoming rancid. Oils are sensitive to heat, light and exposure to oxygen. The use of a vacuum reduces both the contribution of heat and of oxygen to the degeneration of the oil.

In some embodiments the pressure may be increased, thereby increasing the boiling point of the ethanol, thereby requiring a greater degree of heating to evaporate the ethanol.

It will be appreciated that the rate of evaporation may be controlled by increasing and decreasing the pressure within the pressure vessel in which the further solution is contained.

In preferred embodiments the vacuum pressure in the pressure vessel is in the range of 1-20 torr.

### Advantages of the Present Invention

Complexing the curcumin with the marine oil phospholipid improves bioavailability as the curcumin is protected by the complex.
Complexing with the phospholipid prevents separation which is seen when curcumin is simply added in combination with an oil (without complexing).
Sourcing the phospholipid from a marine oil allows a preferred molar ratio of curcumin to lipid in the complex to be achieved.
Using a marine oil sourced phospholipid beneficially maintains the synergistic effect seen between Omega 3 fatty acids with curcumin. Omega 3 fatty acids are present in marine oil phospholipids.
There is good public acceptance and trust of marine oils used in therapeutic compositions.
addition of lecithin in place of some of the marine oil sourced phospholipids beneficially allows one to increase the molar ratio of curcumin to the fatty acids to a particularly preferred ratio (for example, 2.5:1 DHA to curcumin) without a corresponding increase in the viscosity attributed to the fatty acids of the marine oil sourced phospholipids. This may be particularly beneficial for increasing the concentration of curcumin above about 2% w/w in the complex and still be able to formulate into capsules.
Addition of a further diluent in place of some of either the marine oil and/or lecithin may help the formulator to further increase concentration of lecithin upwards of 8%, yet still maintain a usable viscosity for encapsulation methods.
The present invention also provides provision of a stable complex and higher molar ratios of curcumin to fatty acids by increasing the concentration of up to about 20% curcumin. However, in such embodiments, the most preferred option would be tabletting due to the higher viscosity of the composition.

### BRIEF DESCRIPTION OF DRAWINGS

Further aspects of the present invention will become apparent from the following description which is given by way of example only and with reference to the accompanying drawings in which:

FIG. 1 is a flow diagram of one preferred method of preparing a composition in accordance with the present invention, and

FIG. 2 is a further more detailed flow diagram of one preferred method of preparing a composition in accordance with the present invention.

### BEST MODES FOR CARRYING OUT THE INVENTION

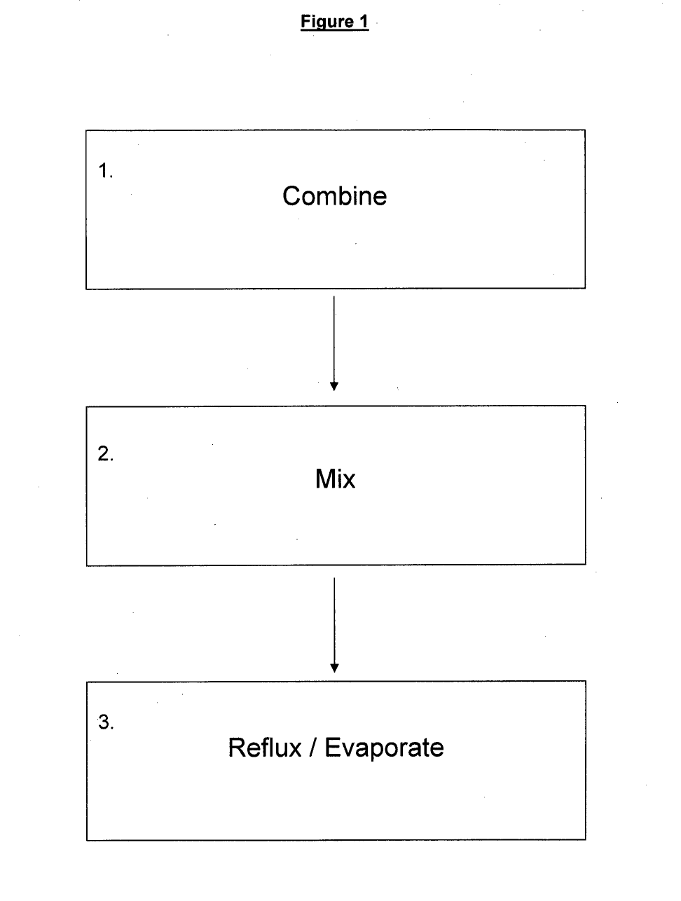

FIG. 1 is a flow diagram illustrating the overall stages of producing a particularly preferred composition in accordance with the present invention. Box 1 of FIG. 1 depicts the stage of combining of the various components such as curcumin, ethanol, marine oil and lecithin. It will be appreciated that combination may be performed in any order without departing from the scope of the invention.

Box 2 of FIG. 1 depicts a mixing step in which the further solution is mixed in order to evenly distribute the different components. It will be appreciated that this step could be combined with the combination step of box 1 and/or the reflux/evaporation of step 3. The mixing step 2 may be performed for a fixed period prior to reflux/evaporation step 3 so as to ensure even mixing.

Box 3 of FIG. 1 depicts a reflux/evaporation step. Reflux/evaporation may be achieved in a number of ways, including:
boiling the mixed further solution at ambient pressure until the solvent has been evaporated off;
boiling the mixed further solution at elevated temperatures at above ambient pressure;
boiling the mixed further solution at reduced temperatures at below ambient pressure.

It will be appreciated that boiling of the further solution may involve boiling of one or more fluids contained in the further fluid. For example the boiling point of the solvent may be substantially lower than that of other fluids in the further fluid, as such the temperature/pressure may be controlled so that only the solvent is boiled off.

The evaporation stage may be controlled either manually, or by way of a control system, to control the rate of evaporation.

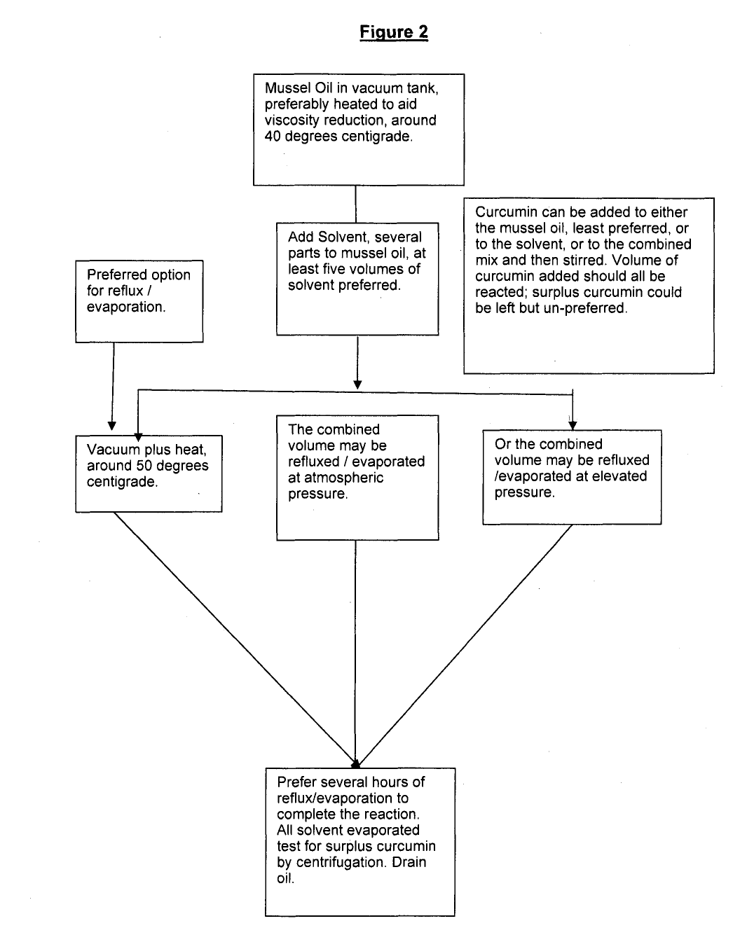

FIG. 2 shows a more detailed flow diagram of the method described above.

Typically the curcumin to be used will be initially as a powder. The powder can be dissolved in the solvent and the resulting solution added to the at least one or more further compounds. If the one or more further compounds include powdered compounds these can also be dissolved in the solvent prior to combining with the other compounds. The solvent may also be combined with a particularly viscous fluid in order to make the viscous fluid more fluent.

In some embodiments a further step may be included which involves taking a sample of fluid during the reflux/evaporation stage and centrifuging the sample to ascertain the degree of bonding between at least the first constituent and at least one of the at least one further constituents. It will be appreciated the in cases where bonding has not occurred centrifuging results in sedimentation of the first constituent forming. Where strong bonding has occurred little or no sedimentation is formed during centrifuging.

### EXAMPLES

The invention is further described by way of reference to the following examples. These examples should not however be construed as being limiting.

### Example 1

### Method of Manufacture

In this example, the amounts of each component added is based on a “by weight” amount as illustrated in Example 2.

Step 1: In a vacuum tank, 40-50 parts of ethanol is mixed with an amount of curcumin, quercetin and piperine.

Step 2: Mixing occurs under reflux and at a temperature of approximately 40-50° C. until all the components are dissolved.

Step 3: An amount of mussel oil and lecithin are first pre-mixed together, and then added to the solution formed by step 2.

Step 4: After 30 minutes of reflux, the mixture from step 3 is further mixed under a slow vacuum at around 40-50° C. for 1-2 hours and then a full vacuum until all the ethanol is boiled off and collected in a evaporator leaving the curcumin bound to the phospholipid as a complex.

### Example 2

### Exemplary Composition of the Present Invention

| Component | Relative amount |
|---|---|
| Demethoxycurcumin | 3.76% w/w |
| Mussel oil | 31.9% w/w |
| Vegetable oil lethicin | 63.9% w/w |
| Piperine | 0.04% w/w |
| Quercetin | 0.2% w/w |
| Total | Approx 100% |

### Example 3

To illustrate the effectiveness of forming the curcumin-phospholipid complex on stability, the following study was done.

A control composition was produced by directly mixing 4% by weight curcumin powder with 500 ml of mussel oil with a blender. The mixture was vigorously mixed in the blender for 20 minutes.

A comparable trial composition (4% curcumin) was then produced according to the method outlined in example 1 (method of manufacture) and 2 (example composition) and in accordance with the present invention.

The control and trial compositions were both placed in an IEC DPR6000 centrifuge and subjected to 5,400G for 1 hour. The resulting precipitate was weighed for each of the control mixtures.

The results showed that the control composition had only 0.3% by weight as soluble curcumin. This correlates to known rates of between 0.2 to 0.5% w/w solubilisation of curcumin. Oppositely, the trial composition did not produce any precipitate indicating that the entire 4% w/w of curcumin had been bound to the phospholipids either from the mussel oil or the lecithin.

### Example 4

The inventors compared a composition having just marine oil to a composition having both marine oil and lecithin. The concentration of curcumin was kept at 4% w/w for both compositions. It was found that the stability of the complex was not affected in the composition having lecithin. This meant that convenient encapsulation techniques could still be achieved for the lecithin-containing composition even when curcumin loading was increased beyond 4% w/w.

Aspects of the present invention have been described by way of example only and it should be appreciated that modifications and additions may be made thereto without departing from the scope thereof.

1. A complex comprising: a phospholipid and curcumin, wherein
some or all of the phospholipid is a marine oil phospholipid.

2. The complex as claimed in claim 1 wherein the marine oil is mussel oil.

3. The complex as claimed in claim 1 wherein the phospholipid includes DHA and/or EPA.

4. The complex as claimed in claim 1 wherein at least a portion of the phospholipid in the complex is phosphatidylcholine.

5. The complex as claimed in claim 1 wherein the complex includes between 1 to 15% w/w curcumin.

6. The complex as claimed in claim 1 wherein the complex includes approximately 2-8% w/w curcumin.

7. The complex as claimed in claim 1 wherein a molar ratio of the curcumin to polyunsaturated fatty acids in the complex is in the range of about 1:2 to 20:1.

8. The complex as claimed in claim 1 wherein a molar ratio of the curcumin to polyunsaturated fatty acids in the complex is in the range of about 1:2 to 5:1.

9. The complex as claimed in claim 1 wherein a ratio of curcumin to phospholipid in the complex is between 1:100 to 1:5.

10. The complex as claimed in claim 1 wherein a ratio of curcumin to phospholipid in the complex is between 1:5 and 1:20.

11. The complex as claimed in claim 1 wherein the marine oil contains greater than 20% w/w phospholipid.

12. The complex as claimed in claim 1 wherein the marine oil contains approximately 40% w/w phospholipid.

13. The complex as claimed in claim 1 wherein an amount of phospholipids in the complex are sourced from lecithin.

14. The complex as claimed in claim 1 wherein the complex also includes piperine and/or quercetin.

15. A composition including the complex as claimed in claim 1.

16. The composition as claimed in claim 15 further including a marine oil.

17. The composition as claimed in claim 15 wherein the composition includes lecithin.

18. The composition as claimed in claim 17 wherein the ratio of lecithin to marine oil in the composition is between 1:3 to 3:1.

19. The composition as claimed in claim 17 wherein the ratio of lecithin to marine oil in the composition is approximately 2:1.

20. The composition as claimed in claim 17 wherein the lecithin is a vegetable oil.

21. The composition as claimed in claim 15 wherein the composition includes a diluent.

22. The composition as claimed in claim 21 wherein the ratio of diluent:lecithin:mussel oil is approximately 1:1:1.

23. The composition as claimed in claim 21 wherein the diluent is oleic acid.

24. The composition as claimed in claim 15 wherein the viscosity of the composition is below 5000 cPs when measured on a Spindle 21 at 35° C. at 1.5 rpm.

25. The composition as claimed in claim 15 wherein the composition includes an added source of polyunsaturated fatty acids DHA and/or EPA.

26. The composition as claimed in claim 15 wherein the composition includes an adjuvant.

27. The composition as claimed in claim 15 wherein the composition includes quercetin.

28. The composition as claimed in claim 15 wherein the composition includes piperine.

29. The composition as claimed in claim 15 wherein the composition is provided in a liquid form in a soft gel capsule.

30. The composition as claimed in claim 15 wherein the composition is provided in a solid dosage tablet form.

31. A method of preparing a complex as claimed in claim 1, the method including the steps of:
a) dissolving a quantity of curcumin in a solvent to form a first solution;
b) mixing the first solution with a quantity of the phospholipid sourced from a marine oil to form a second solution;
c) processing the second solution to form the complex; and
d) separating the complex from the solvent.

32. The method as claimed in claim 31 wherein the solvent is a Protic solvent.

33. The method as claimed in claim 31 wherein solvent is ethanol.

34. The method as claimed in claim 31 wherein step a) includes mixing approximately 40-50 parts volume of solvent to about 1 part curcumin.

35. The method as claimed in claim 31 wherein step a) includes adding quercetin and/or piperine.

36. The method as claimed in claim 31 wherein step b) includes addition of lecithin.

37. The method as claimed in claim 31 wherein step b) includes addition of a diluent.

38. The method as claimed in claim 31 wherein steps a), b) and/or c) include refluxing the solvent.

39. The method as claimed in claim 31 wherein step c) includes applying a slow vacuum at around 40-50° C. for 1-2 hours and then a full vacuum until all the solvent is boiled off from the solution.

40. (canceled)

41. (canceled)
